(19) United States
(12) Patent Application Publication
(10) Pub. No.: US 2015/0050686 A1
(43) Pub. Date: Feb. 19, 2015

(54) OMNIDIRECTIONAL, MULTIAXIAL BIOPRINTED TISSUE SYSTEM, TECHNIQUES AND APPLICATIONS

(71) Applicant: Lawrence Livermore National Security, LLC, Livermore, CA (US)

(72) Inventors: Heeral Sheth, San Francisco, CA (US); Margaret Windy Mcnerney, Pleasanton, CA (US); Satinderpall S. Pannu, Pleasanton, CA (US); Elizabeth K. Wheeler, Livermore, CA (US)

(21) Appl. No.: 14/452,453

(22) Filed: Aug. 5, 2014

Related U.S. Application Data
(60) Provisional application No. 61/865,550, filed on Aug. 13, 2013.

(51) Int. Cl. G01N 33/50

(52) U.S. Cl. CPC G01N 33/5088
USPC 435/29; 435/397

(57) ABSTRACT

A tissue system includes: a support material; and a vascular network comprising a plurality of channels disposed in the support material. A method includes printing a bioink in a support structure to form a network of vascular precursor materials; and converting the vascular precursor materials into a physiologically relevant vascular network. Notably, the tissue systems, networks, etc. are physiologically-relevant, i.e. exhibiting one or more characteristics indicative of physiological relevance, such as a substantially fractal geometry, inter-vessel spacing, cellular composition, dermal structure, concentric multi-layered structure, etc.

### RELATED APPLICATIONS

The present claims priority to U.S. Provisional Application No. 61/865,550, filed Aug. 13, 2013, which is incorporated herein by reference in its entirety.

This application is also related to U.S. Provisional Application No. 61/817,812, filed Apr. 30, 2013, and U.S. patent application Ser. No. 14/265,019, filed Apr. 29, 2014, which are incorporated herein by reference in its entirety.

The United States Government has rights in this invention pursuant to Contract No. DE-AC52-07NA27344 between the United States Department of Energy and Lawrence Livermore National Security, LLC for the operation of Lawrence Livermore National Laboratory.

### FIELD OF THE INVENTION

The present invention relates to cell biology, and more particularly, this invention relates to systems and methods for bioprinting physiologically relevant, three-dimensional tissue systems for, inter alia, tissue engineering and clinical study applications.

### BACKGROUND

The field of microbiology, and particularly tissue engineering, is continuously advancing and incorporating advances in other fields to useful applications such as cell culture. More recent advances include the ability to culture human cells on two and three-dimensional lattices and/or scaffolds to create simplified tissue structures de novo.

These advances have significant applications for the pharmaceutical industry, since the ability to culture human cells and tissues with increasing precision opens new avenues for more efficient and effective clinical studies. Currently, development of new therapeutics takes over a decade and costs are commonly on the billion-dollar scale. Less than 1% of potential new pharmaceuticals reach market and greater than 10% of those that reach market demonstrate serious unanticipated adverse effects that cause market withdrawal and significant costs in litigation.

Moreover, even those few pharmaceuticals, medical devices, treatment procedures, etc. that reach market are often approved pursuant to clinical studies which do not accurately represent the physiological conditions of the corresponding drug, device, or treatment in vivo, because the clinical models and subjects of clinical study are not accurate physiological representations of actual human anatomy. For example, many recent tissue engineering developments focus on either a simplified two-dimensional (2D) or three-dimensional (3D) representation of a tissue system. Cells are typically plated on a two-dimensional surface, which may have a sophisticated geometry such as a bifurcating network of vessels arranged in single plane.

These two-dimensional applications are of limited relevance to clinical studies because the 2D monolayer of tissue formed thereby does not represent true physiological conditions. For example, the monolayer of tissue is limited to a thickness of approximately 200 microns, since diffusion limits and nutrient exhaustion prevent delivery of essential nutrients and evacuation of waste products to/from cells along a path length greater than approximately 200 microns.

In order to overcome this limitation, a vascular network for delivering nutrients and carrying away waste, e.g. as observed in vivo, is necessary. Accordingly, the 2D systems and techniques are not scalable to larger tissue constructs or systems and remain of limited clinical relevance, to great disadvantage of medical professionals and patients alike. In many cases, thick, multilayered tissue systems are needed to adequately represent the complex physiology of human organs in-vivo.

To overcome some of the limitations presented by 2D techniques, some conventional approaches extend to three-dimensional tissue systems. These systems are constructed from a scaffold or lattice. Living cells and the scaffold or lattice are both encapsulated in an extracellular matrix (ECM), and subsequently the lattice material may be dissolved to leave voids representing a simplified, nonphysiological network of channels for exchanging and/or communicating nutrients, waste, signals, drugs, etc. between the tissue system and the external environment.

Disadvantageously, such simplified lattices and scaffolds do not represent physiological conditions observed in vivo. Rather existing 3D systems, such as shown below in FIG. 1D, are characterized either by a substantially planar geometry, e.g. a plurality cell monolayers stacked in a series of parallel planes, or an uncontrolled geometric arrangement. As a result, and exacerbated by the characteristics of the lattice structure, cellular migration and diffusion is limited to the surface area of the lattice construct. This renders existing 3D lattice and scaffold-based tissue constructs impossible or impractical to integrate with physiologically relevant vasculature, limiting clinical applicability of studies conducted using such artificially simplified tissue systems.

Further, several existing bioprinting techniques have been demonstrated and proved capable of forming biological structures similar to those observed in a given channel of a vascular network. These techniques conventionally include either depositing a series of discrete units (e.g. droplets) of cells into a layered structure. Other techniques employ continuous extrusion to similarly form a biological structure by forming successive layers of extruded material(s).

However, these techniques are of limited physiological relevance because the resulting structures do not accurately reflect the complex, bifurcating, three-dimensional network observed in vivo. Rather, these structures represent simplified portions of a vascular network (e.g. one of the main pathways or capillaries in an in vivo vascular network). Forming a complete, physiologically relevant tissue system using the conventional discrete deposit or continuous extrusion techniques is impractical at best, because each “channel” of the network would need to be independently deposited/extruded and cultured, layer-by-layer, and only thereafter could one attempt to fuse or integrate a plurality of such channels into a physiologically relevant network.

Further, each of these conventional techniques is limited to depositing a single type of cell in any given deposit operation (e.g. depositing a single droplet or extruding a single layer of material), which does not readily permit the physiological formation of vascular structures as occurs in-vivo, for example via vasculogenesis and/or angiogenesis.

Currently available cell culture techniques and instrumentation have not demonstrated any such ability to fuse discrete channels into a functioning vascular network, much less a functioning vascular network characterized by a physiologically relevant, three-dimensional arrangement. Moreover, generating a vascular network comprising channels having varied diameter (e.g. to represent the full range of blood vessels in vivo, from large vessels such as main-arterial and venous pathways, e.g. the carotid arteries or jugular veins, all the way down to capillary vessels having a monolayer of endothelial cells defining an outer diameter from about 5 to about 10 microns) would be an overly burdensome, impractical task.

Rather, the conventional extrusion techniques form a cylindrical structure by layer-wise printing of rods of agarose and or cells to form a cylindrical structure (e.g. a single vessel).

Accordingly, it would be of great benefit to provide tissue systems having physiologically relevant, three-dimensional vascular networks, as well as methods of making the same in order to improve availability and physiological relevance of tissue systems for use in clinical studies, transplantation, as well as other medical and research applications that may become apparent to one having ordinary skill in the art upon reading the present descriptions.

### SUMMARY

In one embodiment, a tissue system includes: a support material; and a vascular network comprising a plurality of channels disposed in the support material. The vascular network is physiologically relevant.

In another embodiment, a method includes: printing a bioink in a support structure to form a network of vascular precursor materials; and converting the vascular precursor materials into a physiologically relevant vascular network.

Of course, the presently disclosed inventive concepts are not limited to the summary presented above. Rather, the various embodiments described herein demonstrate exemplary and illustrative features, characteristics, variants, permutations, techniques, etc. falling generally within the scope of the present disclosures and should be considered within the scope of the instant descriptions, along with any equivalents thereof that would be appreciated by a skilled artisan upon reading the present descriptions.

### BRIEF DESCRIPTION OF THE DRAWINGS

FIG. 1A is a simplified schematic of a bioprinting system, according to one embodiment.

FIG. 1B is a simplified schematic of a bioprinting system having printed a rudimentary vascular network, according to one embodiment.

FIG. 1C is a simplified schematic of a physiologically relevant vascular network generated by bioprinting, according to one embodiment.

FIG. 2A shows a simplified schematic head-on view of a multiaxial bioprinting nozzle, according to one embodiment.

FIG. 2B shows a simplified schematic side view of a multiaxial bioprinting nozzle, according to one embodiment.

FIG. 3A shows a schematic cross-sectional view of a bioprinted rudimentary vascular channel, according to one embodiment.

FIG. 3B shows a schematic cross-sectional view of a coaxially-extruded vascular channel, or a bioprinted, self-arranged vascular channel, according to alternative embodiments.

FIG. 4 is a flowchart of a method, according to one embodiment.

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified.

Unless otherwise specifically noted, as used herein the term “substantially” is to be understood to refer to a proximity to a reference point or value, such as a model system, or one or more characteristics of a model system such as physical properties like density, volume fraction, length, diameter, shape, physical configuration or structure, functional properties like activity (electrical, biological, chemical, etc.), one or more characteristics of physiological relevance as described herein, etc. as would be understood by one having ordinary skill in the art upon reading the present descriptions.

In some approaches, the term “substantially” may be understood to refer to a particular feature's proximity being within 90%, preferably within about 95%, more preferably within about 98%, and still more preferably within about 99% of a corresponding value exhibited for a feature of a respective model or reference system.

The following description discloses several preferred embodiments of in-vitro tissue bioreactors and/or related methods.

Physiologically Relevant Vascular Networks

The presently disclosed technology demonstrates a novel system comprising a physiologically relevant vascular network disposed in a support material such as a hydrogel. As understood herein, a “physiologically relevant vascular network” is a network of vascular channels that substantially represents the vascular network observed in human physiology in vivo. In preferred approaches, a physiologically relevant vascular network is characterized by a substantially fractal geometry.

In the particular case of a physiologically relevant vasculature, the fractal geometry preferably represents a recursively bifurcating tree. In some approaches, the physiologically relevant vasculature may be described by a Cantor-bar fractal set, particularly with respect to the branching capillary structures within the vascular network.

In various embodiments, physiological relevance includes a recursively bifurcating network of vascular channels in three-dimensional space. Preferably, with each bifurcation, the number of branches doubles while the length of branches halves. Moreover, the channels are preferably arranged such that a linear distance between any given channel in the network is no more than 200 microns distance away from the nearest neighboring channel in the network. In additional and/or alternative embodiments, channels are preferably arranged such that a linear distance between any given cell and a nearest neighboring channel in the network is less than or equal to about 100 microns.

In more embodiments, a physiologically relevant vascular network includes a plurality of channels, and the plurality of channels include channels having characteristics of large vascular channels, such as main arteries and veins, as well as channels having characteristics of medium-sized vascular channels, such as arterioles and venules, and still further having small vascular channels, such as capillaries.

In still more embodiments, physiologically relevant vascular networks as described herein are characterized by exhibiting substantially similar shear stress on fluid and/or particles (including cells, etc.) passing therethrough as vascular networks observed in human physiology. For example, in arteries, shear stress is in a range from about 0.1 Pa to about 1.0 Pa, and preferably in a range from about 0.3 Pa to about 1.0 Pa.

Poiseuille's Law, shown below in Eqns. 1A and 1B, generally describes the shear stress relationship for a medium flowing through a cylindrical vessel

### Eqns. 1A-1B: Poiseullle's Law for Shear Stress

$$τ = \frac{4 η q}{π {r}^{3}} \qquad 1 A$$

where τ=shear stress, η=medium viscosity, q=measured flow, and r=cavity (e.g. lumen) radius; and

$$γ = \frac{8 {v}_{m}}{d} , \qquad 1 B$$

where γ=shear rate, vₘ=mean flow velocity, and d=cavity (e.g. end-diastolic internal arterial) diameter.

The instant application discloses methods and systems representing physiologically relevant vascular networks by implementing a unique omnidirectional, multiaxial bioprinting technique to assemble biological material into three-dimensional (3D) micro vascular networks. The techniques employ an omnidirectional platform of micro vascular networks that is capable of perfusing thick physiological tissue constructs with appropriate nutrients such as oxygen, metabolic agents, drugs, etc. for long-term survival using a physiologically relevant vascular network as described herein.

The presently disclosed embodiments improve the physiological geometry and omnidirectional interconnectivity of artificially grown vascular networks by implementing a unique bioprinting technique. In addition, the printing and incubation techniques produce conditions in which multi-cellular components may self-assemble and organize into functional tissue, in multiple embodiments.

In one general embodiment, a tissue system includes: a support material; and a vascular network comprising a plurality of channels disposed in the support material. The vascular network is physiologically relevant.

In another general embodiment, a method includes: printing a bioink in a support structure to form a network of vascular precursor materials; and converting the vascular precursor materials into a physiologically relevant vascular network.

Notably, the tissue systems, networks, etc. are physiologically-relevant, i.e. exhibiting one or more characteristics indicative of physiological relevance, such as a substantially fractal geometry, inter-vessel spacing, cellular composition, dermal structure, concentric multi-layered structure, etc. as would be understood by one having ordinary skill in the art upon reading the present descriptions.

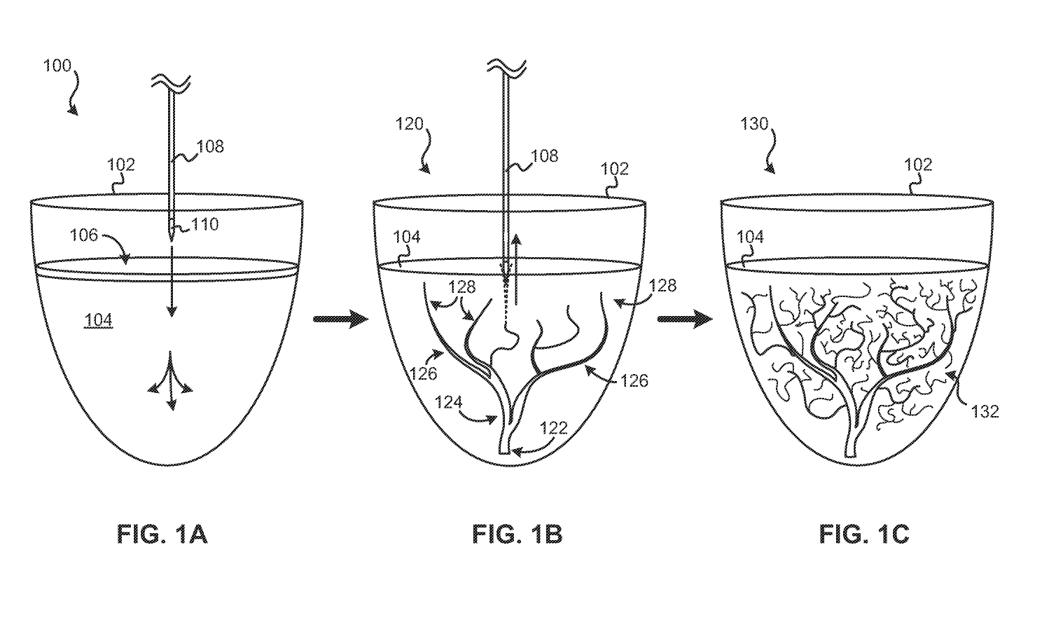

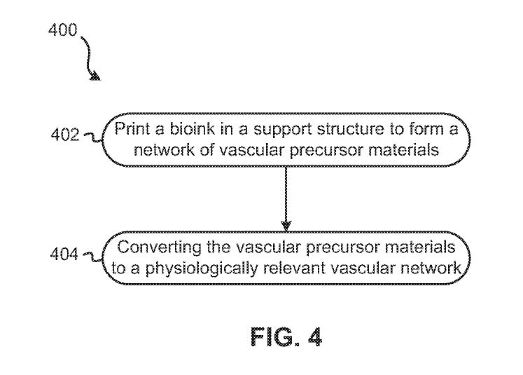

Referring now to the Figures, FIGS. 1A-1C schematically depict a simplified bioprinting system, according to several illustrative embodiments. More specifically, FIG. 1A depicts an illustrative bioprinting system 100 prior to carrying out bioprinting operations to generate, for example, a physiologically relevant vascular network, in one approach. FIG. 1B shows an exemplary embodiment of a rudimentary vascular network 120 characterized by being printed using a bioprinting system and/or bioprinting technique(s) as described herein, e.g. bioprinting system 100 and/or exemplary bioprinting method 400 as shown in FIGS. 1A and 4, respectively. FIG. 1C shows a schematic depiction of a physiologically relevant vascular network 130 characterized by being printed using a bioprinting system and/or bioprinting technique(s) as described herein, e.g. bioprinting system 100 and/or exemplary bioprinting method 400 as shown in FIGS. 1A and 4, respectively.

Referring again to FIG. 1A, the exemplary bioprinting system 100 as described herein includes a container 102, which may be and/or have any suitable material, shape, configuration or characteristics that would be appreciated as advantageous by one having ordinary skill in the art upon reading the present descriptions. Preferably, the container 102 is configured to hold a support material 104 therein without creating any undesirable reaction or conditions in the support material 104. For example, in one embodiment the container comprises a biologically inert material. In additional and/or alternative embodiments, the container 102 is preferably optically transparent to permit facile observation of biological materials/systems printed in the support material 104.

The support material 104 may comprise any suitable material characteristics that would be appreciated as advantageous by one having ordinary skill in the art upon reading the present descriptions. In preferred embodiments, support material 104 is configured to provide three-dimensional structural support to a bioink printed/positioned therein according to the presently described bioprinting techniques. The support material 104 may comprise a matrix substantially representing an extracellular matrix, in preferred embodiments, and more preferably comprises lyophilized, reconstituted human cardiac extracellular matrix and/or components thereof. As described above regarding the container 102, support material 104 is preferably optically transparent to permit facile observation of biological materials/systems printed in the support material 104.

With continuing reference to FIG. 1A, the exemplary bioprinting system 100 also includes a printing mechanism 108, which may include any three-dimensional (3D) printing apparatus suitable for biological printing applications. For example, in some embodiments the printing mechanism 108 may be any known printing device or component thereof capable of being sterilized and operated in a sterile environment without introducing contaminants (e.g. cells, microorganisms, viruses, biological reagents, blood, etc. as would be understood by one having ordinary skill in the art) into the printed material.

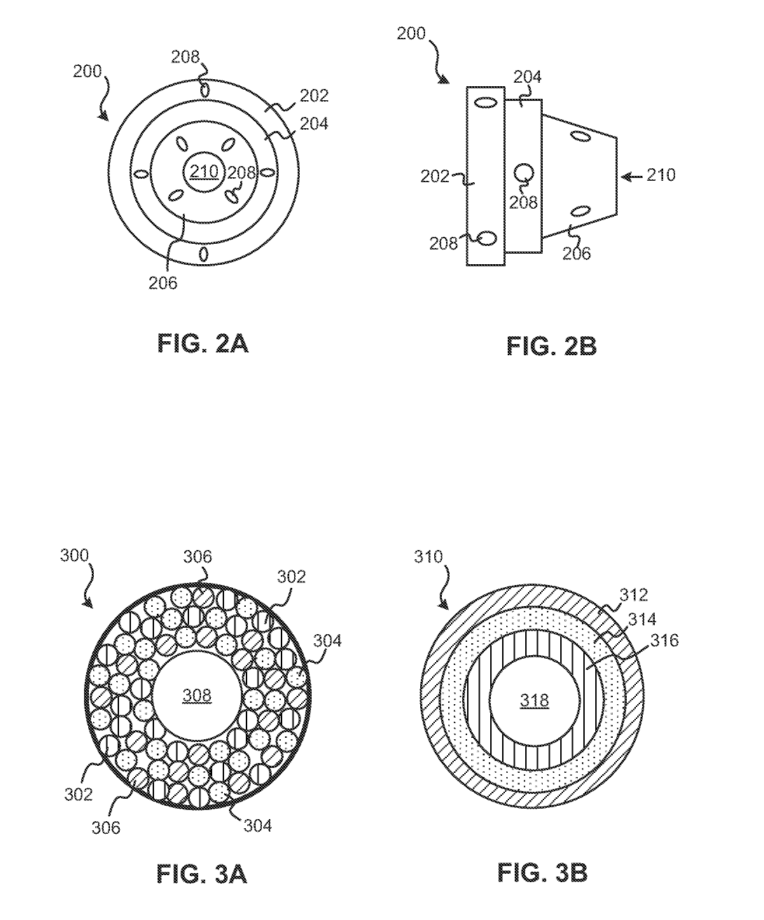

Printing mechanism 108 preferably is an omnidirectional printing device capable of being precisely positioned in a three-dimensional space according to predetermined instructions. The printing mechanism also preferably is capable of traversing the support material 104 without incurring damage to the printing mechanism 108 or any component thereof, in various approaches. Printing mechanism 108 terminates in a nozzle 110, which may be any type of nozzle suitable for use in the presently disclosed applications, techniques, etc., as would be understood by one having ordinary skill in the art upon reading the present descriptions. In preferred embodiments, the nozzle 110 is selected from either a coaxial nozzle or a multiaxial nozzle, with a multiaxial nozzle being particularly preferred. Nozzle types, configurations, and features will be discussed in further detail below with respect to FIGS. 2A-3B, according to some embodiments.

The nozzle is configured to print one or more biological materials, e.g. constituents of a bioink, according to predetermined instructions and generate a network of biological materials in support material 104, e.g. a network of vascular precursor materials such as described below with reference to FIGS. 3A-3B.

Bioprinting system 100 also includes a self-healing fluid 106 disposed in the container 102 and above the support material 104. In operation, the self-healing fluid 106 passively fills void(s) in the support material 104 generated by the printing mechanism 108 during printing. The self-healing fluid 106 may be selected based on the composition of the support material 104, and is generally configured to facilitate structural integrity of the support material 104 throughout a bioprinting process as described herein.

While the self-healing fluid 106 is shown in FIG. 1A as disposed above the support material 104, those having ordinary skill in the art will appreciate that alternative arrangements for support material 104 and self-healing fluid 106 are fully within the scope of the present disclosures. For example, self-healing fluid 106 may be supplied during printing with the bioink material(s) being extruded/deposited, either via a separate nozzle and/or through nozzle 110 in conjunction with the bioink material, in various approaches. Self-healing fluid 106 may also be provided via an external reservoir and supplied to one or more surfaces of and/or locations in the support material 104, in more approaches.

Referring now to FIG. 1B, a rudimentary network 120 of bioprinted material is shown, according to one embodiment. In preferred embodiments, the network 120 is a rudimentary vascular network. As described herein, during printing the printing mechanism 108 travels in up to three dimensions throughout the support material 104, depositing a bioink during motion. Characteristics of the network 120 may be controlled by manipulating one or more of the printing conditions, such as bioink extrusion rate, printing mechanism movement rate and direction, etc.; and/or the physical properties of the materials, e.g. rheological properties of the bioink, structural properties of the support material 104, healing properties of the self-healing fluid 106, etc. as would be understood by those having ordinary skill in the art upon reading the present descriptions.

As shown in FIG. 1B, the exemplary rudimentary network 120 includes a variety of features, such as a base channel 122 that is preferably characterized by a relatively largest size (e.g. inner diameter, wall thickness, etc.) of all channels in the rudimentary network 120. In exemplary embodiments where the rudimentary network 120 represents a rudimentary vasculature, the base channel(s) 122 may be characterized by one or more physical features substantially similar or identical to those observed in vivo for human arteries and large veins. For example, the base channel(s) 122 may have an inner diameter in a range from about 0.1 to about 20 mm, a channel wall about three cell-layers thick, etc. in various approaches and as would be understood by one having ordinary skill in the art upon reading the present descriptions.

The rudimentary network 120 also includes a plurality of bifurcations or branches 124 formed by printing multiple channels so that each channel has a common intersection at the branch or bifurcation point 124. By printing a series of such branches 124, the presently disclosed systems and techniques enable formation of a network of biological materials heretofore unprecedented in terms of physiological relevance.

With continuing reference to FIG. 1B, the rudimentary network 120 generally proceeds from the bottom of container 102 to the top of container 102 with a series of channels characterized by progressively smaller size (e.g. diameter, wall thickness, etc.). In this manner, the rudimentary network 120 is printed with a diverse array of features generally representing some or all of the features observed in vivo for corresponding networks. The printing mechanism may print a channel beginning near the bottom of container 102, and may print one or more channels branching from a previously printed channel to form a bifurcating rudimentary structure such as shown in FIG. 1B, in some approaches.

Upon completing a given printing operation, the printing mechanism 108 optionally evacuates the support material 104 (as indicated by the vertical line pointing upward in FIG. 1B), leaving a void (indicated by dashed lines) that is immediately filled and/or repaired by self-healing fluid 106, in some approaches. The printing mechanism may then reenter the support material 104 and begin printing a new channel de novo in the support material 104 and/or extend a new channel from a previously printed channel. Alternatively, the printing mechanism may terminate printing one channel and begin printing another new or extension channel without evacuating from the support material 104, in more approaches.

The printing mechanism may repeat the process generally described above until the printing operation is complete (e.g. the entire predefined pattern is printed).

For example, one embodiment of an illustrative rudimentary vascular network 120 may include base channels 122 representative of large arteries and/or veins, intervening channels 126 representative of muscular arteries and/or arterioles, venules, medium veins, etc., as well as terminal channels 128 representative of capillaries. The presence and diversity of such features may be controlled as mentioned briefly above by manipulating the properties of the bioink, support material 104, self-healing fluid 106, and/or printing instructions, in various approaches.

The printing mechanism 108, in some approaches, prints a rudimentary network 120 by extruding bioink through nozzle 110 according to a predetermined pattern. The pattern may be user-defined, generated using modeling tools, etc. as would be understood by one having ordinary skill in the art upon reading the present descriptions. In many approaches, the printing mechanism 108 generally penetrates the support material 104 and prints into voids in the immediate proximity of nozzle 110 created by and/or during movement of the printing mechanism 108 throughout support material 104 in up to three dimensions. In preferred approaches, the printing mechanism 108 begins printing near a bottom or base of container 102, and moves in a generally upward direction toward an upper surface of support material 104, gradually tapering or narrowing the size (e.g. inner diameter, wall thickness, etc.) of various channels 122, 126, 128 printed during movement of the printing mechanism 108.

The support material is preferably configured such that the printed bioink is provided structural support in three dimensions without any printed microstructures (e.g. coaxial layers of bioink constituents such as described below with reference to FIGS. 3A-3B, especially interior cavities such as lumen 308, 318) being damaged by the support material 104 and/or self-healing fluid 106 repairing/refilling voids created during the printing operation by printing mechanism 108.

Turning now to FIG. 1C, a physiologically-relevant network 130 of bioprinted materials is shown, according to a simplified schematic. As described above with reference to FIG. 1B, the physiologically-relevant network 130 includes some or all features of a corresponding network as would be observed in vivo. With comparison to the rudimentary network 120 shown in FIG. 1B, the physiologically relevant network 130 is characterized by a greater number of physiologically-relevant features and therefore is more representative of a corresponding in vivo system. For example, the physiologically relevant network may be characterized by an inter-channel spacing 132 substantially representative of a corresponding tissue system as observed in vivo.

In one particular example, and again referring to the human vasculature as a model, the physiologically relevant network 130 shown in FIG. 1C includes a much higher surface area, more channels (especially terminal channels 128) and greater volume fraction occupancy (as well as a more even distribution of volume occupied) of the support material 104, etc. than the rudimentary network 120 shown in FIG. 1B.

In preferred embodiments, the printed bioink, support material 104 and/or self-healing fluid include all constituent materials required to generate a physiologically relevant network (e.g. 130) from a rudimentary network 120. Even more preferably, the rudimentary network 120 will spontaneously generate a physiologically relevant network 130, even if merely provided with nothing more than predetermined time incubation conditions (e.g. temperature, gas composition, humidity, etc. as would be understood by one having ordinary skill in the art upon reading the present descriptions) for a duration referred to herein as an “incubation period,” which may range from several hours to several days or weeks, depending on the types of cell types, tissues, structures, etc. that are to be generated by bioprinting.

For example, in one embodiment printing a rudimentary vascular network 120 may produce cellular structures representative of those observed in vivo for human vascular networks (such as described below with reference to FIGS. 3A-3B) may be observed after as little as 12 hours incubation of the printed rudimentary network at physiological conditions, e.g. 37 centigrade in an approximately 5% CO₂ atmosphere, in some approaches. Even more preferably, the rudimentary vascular network 120 is configured to spontaneously initiate tissue organization and/or generation processes, such as angiogenesis and/or vasculogenesis, to generate a physiologically relevant vascular network 130.

In various embodiments, the presently described networks may include one or more additional and/or alternative features to those described above.

For example, in one approach, a vascular network includes a plurality of constituents selected from a group consisting of endothelial cells (EC), smooth muscle cells, growth factors, and adhesion proteins. These constituents are preferably present in the printed bioink from which rudimentary network 120 is formed.

In various embodiments, the vascular network has physical characteristics of being formed from omnidirectional printing of a bioink, such as presence of a diverse variety of channel types such as described above, a bifurcating structure, inter-vessel spacing similar to that observed in corresponding systems in vivo, etc. as would be understood by skilled artisans upon reading these disclosures.

In one specific embodiment, the physical characteristics of formation from omnidirectional printing include each printed channel being characterized by an outer diameter in a range from approximately 0.5 microns to approximately 1 mm.

Further, the channels may include large or base channels characterized by an outer diameter between about 100 microns and about 20 mm: medium or intervening channels characterized by an outer diameter between about 7 microns and about 150 microns; and terminal or capillary channels characterized by an outer diameter between about 5 microns and about 40 microns, in various approaches.

The channels may be even further characterized by an inter-channel spacing between approximately 0.01 microns and approximately 200 microns.

In some approaches, the bioink (and thus the constituents described above) may alternatively and/or additionally include a fugitive material configured to vacate an interior cavity of some or all channels of the rudimentary network. Preferably, fugitive materials as described herein are configured to vacate the interior cavity in response to exposure to predetermined conditions, such as exposure to a solvent, passage of a predetermined period of time, etc. as would be understood by one having ordinary skill in the art upon reading the present descriptions.

In further embodiments, the support material 104 may include one or more of the following constituents: MATRIGEL™ Stock, MATRIGEL™/GM mixture, EXTRACELL™, PURAMATRIX™, Agarose, Sodium alginate/Calcium (II) chloride, Collagen (Types I-IV), lyophilized/reconstituted human cardiac ECM, gelatin, polyethylene glycol (PEG), polyethylene glycol diacrylate (PEGDA), and/or poly-L-lactic acid (PLLA), a buffer such as phosphate-buffered saline (PBS), and/or one or more cell-type specific culture growth media.

Referring now to FIGS. 2A-2B, one exemplary embodiment of a multiaxial nozzle 200 are shown, according to a simplified schematic depicted from a front and side view, respectively. The multiaxial nozzle 200 is but one example of a suitable nozzle type that may be used as nozzle 110 of bioprinting system 100, in various approaches. Those having ordinary skill in the art will appreciate upon reading the present descriptions that any suitable nozzle type may be utilized without departing from the scope of the present descriptions, including but not limited to a simple nozzle (e.g. single-channel nozzle), coaxial nozzle, etc. Moreover, multiple nozzles may be utilized in unison without departing from the scope of the instant disclosures, in more embodiments.

As shown according to the schematic front-view in FIG. 2A, the exemplary multiaxial nozzle 200 includes a plurality of stages 202, 204, 206 arranged in a concentric manner around a central aperture 210. Each stage 202, 204, 206 is generally circular in cross-sectional profile, and is configured to extrude one or more bioink constituents therethrough according to predetermined conditions (such as flow rate, shear stress, deposition rate/amount/size), etc. as would be understood by one having ordinary skill in the art upon reading the present descriptions.

The stages 202, 204 and 206 are arranged such that the profile of each stage decreases in diameter in a direction from a rear of the nozzle 200 to the front of the nozzle 200 where central aperture 210 is positioned, in one embodiment. Each stage 202, 204, 206 may have any suitable configuration, and as shown in the representative embodiment of FIGS. 2A-2B, outer stage 202 and intermediate stage 204 each terminate in a roughly cylindrical configuration, while central stage 206 terminates in a shape representing a truncated cone.

Preferably, one or more of the stages 202, 204 and 206 include at least one auxiliary aperture 208 in addition to the central aperture 210 in central stage 206. Even more preferably, each of the stages may rotate around a central axis (extending through an interior of the nozzle 200 and out of the central aperture 210 in a direction generally indicated by the arrow of reference numeral 210 shown in FIG. 2B) independently, such that each stage may be rotated during printing in a unique manner to generate customized macro and/or microstructures within a printed rudimentary network 120, in some approaches. Each stage 202, 204, 206 may be independently rotated with respect to both direction and speed, in preferred embodiments.

Further still, in some approaches one or more bioink constituents may be interchangeably and independently or cooperatively extruded/printed through various apertures 208, 210 of the multiaxial nozzle 200 to precisely control the configuration of the resulting rudimentary network 120.

Turning now to FIGS. 3A-3B, various embodiments of an exemplary printed channel microstructure will be discussed. According to the cross-sectional schematic shown in FIGS. 3A and 3B, two alternative exemplary configurations of a printed channel microstructure 300, 310 may be alternatively and/or cooperatively employed in various approaches to bioink printing techniques described herein.

As shown in FIGS. 3A-3B, a bioink as described herein may be extruded in one or more concentric layers, and each layer may comprise one or more constituents. For example, as shown in FIG. 3A, the bioink was extruded through a nozzle, e.g. a coaxial nozzle, to deposit a mixed population of cell types and/or tissue precursors in a commingled layer. According to the exemplary embodiment shown therein, the mixed cell population includes three separate cell types 302, 304, 306 that are preferably precursors configured to facilitate and/or completely control formation of a physiologically relevant tissue structure and/or physiologically relevant network, e.g. physiologically relevant network 130 as shown in FIG. 1C. The cell types 302, 304, 306 form a cylindrical wall structure separating an external environment (e.g. support material 104) from an interior cavity 308 of the printed channel.

In a preferred embodiment, the cell types 302, 304, 306 include endothelial cells, smooth muscle cells, and fibroblasts surrounding the interior cavity or lumen 308. Even more preferably, the endothelial cells, smooth muscle cells, and fibroblasts are configured to and supplied with all materials necessary to generate a physiologically relevant channel structure from the printed structure of mixed cell populations (e.g. via vasculogenesis), and/or a physiologically relevant network from the printed structure of mixed cell populations (e.g. via angiogenesis following formation of the specialized vascular tissue structure via vasculogenesis as described above).

In additional approaches, the interior cavity 308 may alternatively comprise a fugitive material configured to evacuate the volume occupied thereby under predetermined conditions as described above, or an interior void space having characteristics as described herein (e.g. channel size, diameter, wall thickness, etc. as would be understood by one having ordinary skill in the art upon reading the present descriptions).

The inventors have observed self-organization of vascular tissue structures representative of those observed in vivo from co-axially printed populations of cell types such as shown in FIG. 3A and described above. Characterization of such tissues revealed physiologically-relevant characteristics thereof, confirming the capacity to form mature, model systems representative of corresponding tissue systems in vivo for a variety of useful applications.

Now with reference to FIG. 3B, another exemplary printed structure 310 is shown according to one embodiment. According to the cross-sectional view depicted in FIG. 3B, the exemplary structure 310 includes a plurality of layers 312, 314, 316 arranged in a concentric fashion around a central cavity 318. The cross-sectional structure 310 may be obtained, in some approaches, by printing bioink as described generally above using a multiaxial nozzle such as multiaxial nozzle 200 shown in FIGS. 2A-2B and described above. Advantageously, using a multiaxial nozzle may enable printing of organized structures substantially similar to those observed in vivo for a corresponding tissue system.

For example, and again referring to FIG. 3C, the exemplary structure 310 comprises concentric layers 312, 314, 316 surrounding interior cavity 318. In a particularly preferred embodiment designed to represent human vasculature, layer 312 comprises fibroblasts and is disposed radially around a periphery of the structure 310, layer 314 comprises smooth muscle cells and is disposed interior to layer 312 and exterior to layer 316, and layer 316 comprises endothelial cells, which are in turn is disposed radially around a periphery of the interior cavity 318 and interior to layer 314. Interior cavity 318 again comprises either a fugitive material or an internal void, alternatively, in some approaches.

Referring now to FIG. 4, an exemplary method 400 for making a physiologically relevant vascular network generally according to the principles described herein is presented, according to one illustrative embodiment. As will be appreciated by one having ordinary skill in the art upon reading the present descriptions, the method 400 may be carried out in any suitable environment, including those depicted in FIGS. 1-3B, among others.

As shown in FIG. 4, method 400 includes operation 402, where a bioink is printed in a support structure to form a network of vascular precursor materials.

In addition, method 400 includes operation 404, where the vascular precursor materials are converted into a physiologically relevant vascular network.

The printing, in some approaches, may include multiaxial extrusion of the bioink through a nozzle. In more approaches, the printing may include omnidirectional printing.

In various approaches, the printing forms the vascular network in a geometric arrangement characterized by an inter-channel spacing between approximately 1 micron and approximately 175 microns.

In more approaches, incubating the support structure and the bioink under physiological conditions for a predetermined duration may be performed, e.g. to improve the physiological relevance of the vascular network.

Still more approaches may include characterizing one or more tissues of the vascular network. The characterizing may include one or more of: optical imaging techniques, fluorescent imaging techniques, radiological imaging techniques, measuring tissue response to one or more compounds; and measuring tissue response to one or more stimuli.

Additional and/or alternative approaches may further include removing waste from one or more of: tissues and/or cells in the vascular network; and tissues and/or cells proximate to the vascular network (e.g. within 0.01-300 microns linear distance from a venous vascular channel). These approaches may also include providing nutrients to one or more of: tissues and/or cells in the vascular network; and tissues and/or cells proximate to the vascular network (e.g. within 1-300 microns linear distance from an arterial vascular channel).

Various additional and/or alternative features, techniques, and/or structures in addition to those described herein are also within the scope of the presently disclosed systems and methods, which are disclosed more fully in related U.S. Appl. Nos. 61/856,550, filed Aug. 13, 2013, and 61/817,812, filed Apr. 30, 2013, which were previously incorporated by reference above.

While various embodiments have been described above, it should be understood that they have been presented by way of example only, and not limitation. Thus, the breadth and scope of an embodiment of the present invention should not be limited by any of the above-described exemplary embodiments, but should be defined only in accordance with the following claims and their equivalents.

What is claimed is:

1. A tissue system, comprising:
a support material, and
a vascular network comprising a plurality of channels disposed in the support material, wherein the vascular network is physiologically relevant.

2. The system as recited in claim 1, wherein the vascular network comprises a plurality of constituents selected from a group consisting of endothelial cells (EC), smooth muscle cells, growth factors, and adhesion proteins.

3. The system as recited in claim 2, further comprising a fugitive material configured to vacate an interior cavity of each channel in the vascular network in response to exposure to predetermined conditions.

4. The system as recited in claim 1, wherein the support material comprises one or more of: MATRIGEL™ Stock, MATRIGEL™/GM mixture, EXTRACELL™, PURAMATRIX™, Agarose, Sodium alginate/Calcium (II) chloride, Collagen (Types I-IV), lyophilized/reconstituted human cardiac ECM, gelatin, polyethylene glycol (PEG), polyethylene glycol diacrylate (PEGDA), and/or poly-L-lactic acid (PLLA), a buffer such as phosphate-buffered saline (PBS), and/or one or more cell-type specific culture growth media.

5. The system as recited in claim 1, wherein the vascular network has physical characteristics of being formed from omnidirectional printing of a bioink.

6. The system as recited in claim 1, wherein the vascular network comprises arterial pathways and venous pathways.

7. The system as recited in claim 1, each channel being characterized by an outer diameter in a range from approximately 0.5 microns to approximately 1 mm.

8. The system as recited in claim 1, wherein the channels comprise one or more of:
large channels characterized by an outer large channel diameter between about 100 microns and about 20 mm;
medium channels characterized by an outer medium channel diameter between about 7 microns and about 150 microns; and
capillary channels characterized by an outer capillary diameter between about 5 microns and about 40 microns.

9. The system as recited in claim 1, wherein the vascular network is characterized by an inter-channel spacing between approximately 0.01 microns and approximately 200 microns.

10. The system as recited in claim 1, wherein the vascular network comprises a bifurcating network of the channels.

11. The system as recited in claim 1, wherein the vascular network has physical characteristics of being formed at least in part by vasculogenesis and/or angiogenesis.

12. A method, comprising:
printing a bioink in a support structure to form a network of vascular precursor materials; and
converting the vascular precursor materials into a physiologically relevant vascular network.

13. The method as recited in claim 12, wherein the printing comprises multiaxial extrusion of the bioink through a nozzle.

14. The method as recited in claim 12, wherein the printing comprises omnidirectional printing.

15. The method as recited in claim 12, wherein the printing forms the network in a geometric arrangement characterized by an inter-channel spacing between approximately 1 micron and approximately 175 microns.

16. The method as recited in claim 12, further comprising: incubating the support structure and the bioink under physiological conditions for a predetermined duration.

17. The method as recited in claim 16, further comprising: characterizing one or more tissues of the vascular network.

18. The method as recited in claim 17, wherein the characterizing comprises one or more of:
optical imaging techniques, fluorescent imaging techniques, radiological imaging techniques, measuring tissue response to one or more compounds; and measuring tissue response to one or more stimuli.

19. The method as recited in claim 12, further comprising removing waste from one or more of:
tissues and/or cells in the vascular network; and
tissues and/or cells proximate to the vascular network.

20. The method as recited in claim 12, further comprising providing nutrients to one or more of:
tissues and/or cells in the vascular network; and
tissues and/or cells proximate to the vascular network.
